(19) United States
(12) Patent Application Publication
(10) Pub. No.: US 2024/0148621 A1
(43) Pub. Date: May 9, 2024

(54) COSMETIC COMPOSITION CONTAINING AT LEAST ONE COSMETIC POWDER WITH CHROMOPHORE-BASED SURFACE COATING

(71) Applicant: INTERCOS S.p.A., Milano (IT)

(72) Inventors: Claudio PIROVANO, VERDERIO (LC) (IT); Patrizia VALSESIA, CALCO (LC) (IT)

(73) Assignee: INTERCOS S.p.A., Milano (IT)

(21) Appl. No.: 18/279,445

(22) PCT Filed: Feb. 25, 2022
(86) PCT No.: PCT/EP2022/054800

§ 371 Date: Aug. 30, 2023

(30) Foreign Application Priority Data
Mar. 1, 2021 (IT) 102021000004730

(51) Int. Cl. A61K 8/25; A61K 8/02; A61K 8/29; A61Q 1/06; A61Q 1/08; A61Q 1/10; A61Q 5/02; A61Q 19/00

(52) U.S. Cl. CPC A61K 8/25; A61K 8/0225; A61K 8/29; A61Q 1/06; A61Q 1/08; A61Q 1/10; A61Q 5/02; A61Q 19/00; A61K 2800/43; A61K 2800/436; A61K 2800/624; A61K 2800/94

(57) ABSTRACT

A powder cosmetic composition with a chromophore-based coating is described. The composition comprises from 0.1% to 50% by weight of at least one chromophore selected among polymeric chromophores chemically linked to polyols with reactive functional groups and from 99.9% to 50% by weight of a cosmetic powder or mixture of cosmetic powders having superficial reactive groups.

The present invention relates to a cosmetic composition containing at least one cosmetic powder provided with a chromophore-based surface coating which has the function of enhancing and personalizing color.

Fundamental in cosmetics in general, color plays a crucial role in particular in make-up, where it represents the real essence.

Since ancient times, color applied to the body has been and is used not only to enhance the beauty thereof, but also to communicate and arouse emotions.

Therefore, the study and research on color in a broad sense and the expression thereof in make-up products always remain strategic and priority in innovation in cosmetics. This involves, in addition to a continuous study of color trends, also a continuous search for coloring materials, synthesis and formulation techniques aimed at conveying color in the cosmetic product and on the skin in the most vivid and innovative manner.

The coloring materials accepted and suitable for use in cosmetics can be divided mainly into two classes, depending on whether they are soluble or insoluble in the solvents used in the art.

Soluble dyes are organic chromophore molecules of natural or synthetic origin, which are soluble in water, in oil or possibly other solvents. The ingredients are adapted to obtain bright and brilliant colors, avoiding the problems of sedimentation in fluid formulations typical of insoluble pigments, but having the great limitation linked to the stability and tendency to stain the skin in a persistent and undesired manner (phenomenon known as “staining”).

Insoluble dyes, on the other hand, are inorganic pigments mainly consisting of oxides or inorganic salts which are optically transparent (for example mica) or capable of manifesting color through the absorption of specific portions of the visible spectrum (for example iron oxide, titanium dioxide). They are pigments widely used in make-up due to the high opacity or covering capacity thereof. These are opaque colors which are very stable to light and heat, but lacking brightness and intensity.

A particular category of insoluble pigments is that of pearlescent pigments in which the color, depending on the composition of the material, arises from phenomena of interaction with light such as reflection, refraction, interference and transmission, as well as absorption.

Lacquers are the insoluble form of organic dyes. They are obtained by absorption, precipitation or chemical combination of soluble dyes on a substrate, usually aluminum, calcium or barium hydroxide.

Lacquers generally offer a more vivid and brighter color with respect to inorganic pigments and a good stability, and are thus greatly used in cosmetics.

The solubility of organic lacquers is limited at room temperature, with neutral pH and in the absence of other salts. However, at pHs other than neutrality, at high temperature or in the presence of other solutes (salts dissociated in water, for example CACl₂), part of the pigment can dissolve, coloring the aqueous phase, with the generation of a solution of the coloring compound. Such an effect can cause skin coloring due to the absorption of the dye in the skin and product unevenness due to the migration of the die (“bleeding” phenomenon).

Therefore, cosmetic dyes are overall enclosed in a heterogeneous set of materials which have very different nature and chemical-physical features, which have strengths and drawbacks in the application in a product which cannot be standardized in a single scenario. This requires a specific study every time it is necessary, for example, to work on the combination or replacement of dyes from different families to obtain the product with the desired color.

In make-up, the work of developing a formula consists in outlining the base structure thereof in various colors and visual effects (shades) in order to satisfy market trends and meet the needs of consumers with different aesthetic tastes and physical features.

While the ingredients of the base structure of a formula give the finished product the features thereof, defining the texture and main cosmetic performance thereof, the color package obtained by virtue of the combination of different types of dye represents the variable part of the formula.

Obtaining products with different visual effects involves the use of dyes which, as seen above, can have very different chemical-physical properties and involve, in use, different problems which must be taken into consideration.

Furthermore, the different chemical-physical nature means that the various combinations of dyes behave in a totally different manner in formulation, for example in terms of sensoriality, compatibility with other ingredients or dispersion capacity in certain fluid phases, influencing the main features of the basic formula. All this complicates the development of a formula in different colors, making it more difficult to maintain the texture and the main cosmetic performance without changing the base structure thereof.

One of the techniques commonly used in the art in order to improve the cosmetic performance of powders (pigments and fillers) is the surface coating process, according to which the chemical-physical properties of the powders themselves are modulated by chemically modifying the surface. Polymers or molecules with specific properties and with groups capable of forming covalent bonds with the surface of the pigments are used as coating agents. Depending on the nature of the coating agent used, the surface treatment of the powders can modify the wettability, oil absorption, dispersibility and hydrophobicity of the powders, with the aim of having:
better pigment dispersibility and more stable dispersions
better compatibility between pigments of different nature
better compatibility between pigments and ingredients in the formulation
long-lasting make-up
better sensoriality

WO 2007/021731 A2 discloses functionalized pigments which are formed by attaching a dye to the surface of a metal oxide or semi-metal oxide particle using a multifunctional coupling agent. The multifunctional coupling agent bonds with both a surface hydroxyl group of the particle and areactive moiety of the dye, thereby imbuing the pigment particle with the desired color properties of the dye while retaining the desired physical properties of the pigment particle.

WO 2011/149870 A1 discloses the use of polymeric dyes in detergents which do not have cosmetic properties and are not intended for cosmetic use.

The purpose of the present invention was to explore a function of the surface coating of cosmetic powders not yet investigated, i.e., acting directly on the color of the final formulation, with the possibility of enhancing and personalizing it.

A cosmetic composition with coated powder was thus identified in which the powder is characterized in that it comprises:
a) from 0.1% to 50% by weight of at least one chromophore selected among polymeric chromophores chemically linked to polyols with reactive functional groups;
b) from 99.9% to 50% by weight of a cosmetic powder or mixture of cosmetic powders having superficial reactive groups;
wherein said chromophore is chemically linked to the surface of said cosmetic powder or mixture of cosmetic powders.

The direct chemical linking of a polymeric dye having a chromophore chemically linked to polyols on the surface of a cosmetic powder provides advantages with respect to a system in which a chromophore molecule is attached to a pigment by means of a separate coupling agent.

In particular, the cosmetic chromophore acts on both the color and the chemical-physical properties of the powder. The polymeric part, according to the nature of the polymer, acts as a wetting helping the insertion of the modified powder in specific cosmetic formulations.

On the technologic point of view, there is the advantage that the polymeric chromophores do not penetrate into the skin so that they can be considered materials at no risk and allow a much surer profile.

The use of a polymeric chromophore further allows a more homogeneous coating and a higher coloring power.

It is particularly interesting that the chromophores consist of polymeric chromophores chemically linked to polyols, which can be defined as long-chain compounds with a polymeric structure which ends with at least two hydroxyl groups.

Their composition allows their use, for example, in the polyurethane synthesis reactions. Moreover, they can be applied for coloring polymers and thermosetting resins.

It is to be evidenced that the selection of polyols is crucial for the obtainment of colored powders in cosmetics. Chromophores chemically linked to polyols have resulted the most suitable to obtain powders with unique and enhanced colors at the same time capable of maintaining features of writing, skin adhesion and insertion facility in every category of cosmetic product.

Polyols usable for the present invention may be polyesters, polyalkylene oxides (for example, polyethylene oxide, polypropylene oxide, polybutylene oxide), polyglycerols, aliphatic polyols, polysiloxanes, polyols derived from sugar and polysaccharides, polycarbonates, polyacrylates and so on.

The reactive functional groups can be selected among alkoxy groups (for example, —O—CH₃, —O—CH₂, CH₃, —O—CH(CH₃)₂), Si—H, carboxylic groups and others.

The chromophore group can be selected among the main chromophore groups known in the world of organic dyes such as azo groups, anthraquinones, indigoids, polymethines (e.g., cyanine), phthalocyanines, aryl-carbon, trialyl methane).

The coating chromophore a) can be advantageously synthesized from commercially available reagents, for example by reacting the compound 3-isocyanate propyl triethoxy silane (CAS #24801-88-5, available among others from Wako Pure Chemical Industries, Ltd., Osaka, Japan, TCI Europe NV, Zwijndrecht, Belgium or Gelest, Inc. Morrisville, PA, United States) with a reactive polymeric chromophore of the Reactint family, marketed by Milliken, in an equimolar quantity of the reactive groups of the two reagents. The reaction is carried out in ethyl acetate at the temperature of 75° C. for 6 hours and in the presence of a suitable catalyst for the addition reaction.

The powder or mixture of cosmetic powders b) comprises a powder which has, for example, hydroxyl groups on the surface adapted to be functionalized through the formation of stable covalent bonds with the chromophore.

The grafting of the coating on the powder substrate allows selecting a large variety of cosmetic powder substrates with surface hydroxyls, such as silicates, silica, alumina, aluminum hydroxide, titania, iron oxides, glass and especially pigments, in particular pearlescent pigments, and to functionalize them through the formation of stable covalent bonds with the coating agent.

Preferably, the coating process is carried out via sol-gel chemistry by means of the reaction of the reactive triethoxy silane group of the chromophore with the surface of the cosmetic powder (up to 3 covalent bonds per coating molecule).

The cosmetic composition comprising the powder with a formula chromophore base can be used for various types of cosmetic products, for example anhydrous or poured make-up products, water-based products, shampoos, emulsions, balms, powder eye shadows, pencils and products for skin and hair care.

The possibility of coating the same starting cosmetic powder with different chromophores allows obtaining, depending on the selected chromophore, powders with different colors. The common organic nature of the selected chromophore groups is capable of keeping the chemical-physical properties of the powder uniform when the type of chromophore varies, with the exception of the color.

The chemical coating further avoids the solubility problems typical of soluble dyes which lead to staining on the skin and bleeding in the product.

Thereby, the color development process of a formula is significantly simplified and standardized, with an increase in the amount of visual effects (shade) and finishes obtainable with the same chemical-physical properties of the color package, without the need to correct the base structure of the formula in order to maintain the texture and cosmetic properties unaltered. Therefore, this also entails a considerable time optimization of the color development process.

The cosmetic composition containing the powder with a chromophore-based coating can benefit from the ability of the coating to modify some of the main color features of the starting cosmetic powder, such as saturation (chroma) and hue.

The starting cosmetic powder appropriately treated according to the coating presented in the invention can assume a more intense color, increasing the saturation and thus acting as a booster of the starting color. At the same time, it is also possible to modify the coordinates related to the hue, with the possibility of obtaining colors with highly customizable features which cannot be obtained either from the starting powder or from any mixtures of the powder itself with other cosmetic coloring substrates.

### EXAMPLES

### Example 1

Preparation of a “Coating phase with chromophore group”

| | Name | % (w/w) |
|---|---|---|
| Phase A | Ethyl Acetate | 50.000 |
| | Reactint Red X64 | 28.000 |
| | Hexanoic acid, 2-ethyl-, zinc salt, basic | 0.050 |
| Phase B | 3-(triethoxysily1)propylisocyanate, 95% | 20.950 |
| Phase C | Ethanol | 1.000 |

The present coating phase with chromophore group is prepared by putting Phase A in a reactor, provided with a stirrer, thermometer and condenser. Phase B is added at 70° C. under a nitrogen flow and the mass is heated at a temperature of 90° C. for about 10 hours until the active isocyanate groups disappear. This disappearance is evaluated with IR. Phase C is then added at about 70° C.

The pigment selected for this example is in no way limiting with respect to all the other chromophores belonging to the Reactint family, produced by Milliken.

### Example 2

Preparation of a cosmetic powder
with a chromophore-based coating

| | Name | % (w/w) |
|---|---|---|
| Phase A | Mica | 89.500 |
| Phase B | Hydrochloric acid solution (1.2N) | 1.500 |
| Phase C | Coating phase as in example 1 | 9.000 |

The cosmetic powder with a chromophore-based coating of the example was obtained by loading Phase A into a mixer and stirring it with a suitable impeller, Phase B and Phase C were sequentially atomized onto the Phase A powder at room temperature. The wet powder is then discharged into suitable containers and dried in the oven at 80° C. for 24 hours. The volatile content after oven treatment must be <1%. Finally, the powder is sieved with a 120 mesh sieve.

The percentage of Phase C with respect to Phase A can be modulated as a function of the desired color intensity, as long as the evaluated coating reaction is completed. The quantity of unreacted coating on a powder is evaluated by Soxhlet extraction with THF.

The powder selected for this example is in no way limiting with respect to all the other cosmetic powders which can be used (Talc, Silica, etc.).

### Example 3

Preparation of a cosmetic pigment
with a chromophore-based coating

| | Name | % (w/w) |
|---|---|---|
| Phase A | Titanium dioxide | 89.500 |
| Phase B | Hydrochloric acid solution (1.42N) | 1.500 |
| Phase C | Coating phase as in example 1 | 9.000 |

The cosmetic pigment with a chromophore-based coating of the example was obtained by loading Phase A into a mixer and stirring it with a suitable impeller, Phase B and Phase C were sequentially atomized onto the Phase A pigment at room temperature. The wet powder is then discharged into suitable containers and dried in the oven at 80° C. for 24 hours. The volatile content after oven treatment must be <1%. Finally, the pigment is sieved with a 120 mesh sieve.

The percentage of Phase C with respect to Phase A can be modulated as a function of the desired color intensity, as long as the evaluated coating reaction is completed. The quantity of unreacted coating on a powder is evaluated by Soxhlet extraction with THF.

The pigment selected for this example is in no way limiting with respect to all the other cosmetic pigments which can be used (Red iron oxide, Yellow iron oxide, Black iron oxide, etc.).

### Example 4

Preparation of a pearlescent cosmetic pigment
with a chromophore-based coating

| | Name | % (w/w) |
|---|---|---|
| Phase A | Mica and Titanium dioxide | 89.500 |
| Phase B | Hydrochloric acid solution (1.42N) | 1.500 |
| Phase C | Coating phase as in example 1 | 9.000 |

The pearlescent cosmetic pigment with a chromophore-based coating of the example was obtained by loading Phase A into a mixer and stirring it with a suitable impeller, Phase B and Phase C were sequentially atomized onto the Phase A pigment at room temperature. The wet powder is then discharged into suitable containers and dried in the oven at 80° C. for 24 hours. The volatile content after oven treatment must be <1%. Finally, the pigment is sieved with a 90 mesh sieve.

The percentage of Phase C with respect to Phase A can be modulated as a function of the desired color intensity, as long as the evaluated coating reaction is completed. The quantity of unreacted coating on a powder is evaluated by Soxhlet extraction with THF.

The pigment selected for this example is in no way limiting with respect to all the other cosmetic pigments which can be used (pearlescent glass-based pigment, etc.).

### Example 5

Preparation of a lipstick with pearlescent
pigment coated as in example 4

| | Name | % (w/w) |
|---|---|---|
| Phase A | Polyethylene | 7.000 |
| | Caprylic/Capric triglyceride | 8.080 |
| | Pentaerythrityl Tetra-di-t- | 0.020 |
| Phase B | Caprylic/Capric triglyceride | 10.000 |
| | Microcrystalline Wax | 5.000 |
| | Polybutene | 31.000 |
| Phase C | Disteardimonium Hectorite | 0.700 |
| | Ethanol | 0.500 |
| | Caprylic/Capric triglyceride | 11.400 |
| Phase D | Pearlescent pigment coated as in example 4 | 26.300 |

### Example 6

Preparation of an emulsion blush with cosmetic powder coated as in
example 2 and with pigment coated as in example 3

| | Name | % (w/w) |
|---|---|---|
| Phase A | Water | 64.800 |
| | Butylen Glycol | 6.270 |
| | Bis-PEG 18 Methyl Ether Dimethyl Silane | 0.500 |
| | Sodium Dehydroacetate | 0.050 |
| Phase B | Xanthan Gum | 0.030 |
| | Cellulose Gum | 0.040 |
| Phase C | Potassium Cetyl Phosphate | 0.500 |
| Phase D | Pigment coated as in example 3 | 5.400 |
| | Silica | 2.000 |
| | Cosmetic powder coated as in example 2 | 2.600 |
| Phase E | Sodium Acrylate/Sodium Acryloyldimethyl | 2.800 |
| | Taurate Copolymer | |
| Phase F | Beheneth-30 | 1.300 |
| | Cethyl Alcohol | 1.000 |
| | Glyceryl Stearate and Sodium Stearate | 0.600 |
| | Hexyldecanol | 0.600 |
| | Glyceryl Isostearate | 0.600 |
| | Cetearyl Glucoside and Cetearyl Alcohol | 0.200 |
| | Cetearyl Exthylhexanoate | 7.830 |
| | Tocopherol | 0.020 |
| | Ascorbyl Palmitate | 0.020 |
| Phase G | Water | 2.000 |
| | Tetrasodium EDTA | 0.010 |
| Phase H | Caprylyl Glycol and Phenoxyethanol and | 0.500 |
| | Hexylene Glycol | |
| | Phenoxyethanol | 0.330 |

### Example 7

Preparation of a powder eyeshadow with cosmetic powder coated
as in example 2 and with pigment coated as in example 3

| | Name | % (w/w) |
|---|---|---|
| Phase A | Cosmetic powder coated as in example 2 | 36.900 |
| | Zinc Stearate | 6.000 |
| | Silica | 18.000 |
| | Pigment coated as in example 3 | 35.000 |
| Phase B | Caprylic/Capric triglyceride | 3.100 |
| | Ethylhexyl Glycerin | 0.500 |
| | Caprylyl Glycol | 0.500 |

### Example 8

Preparation of a pencil with pigment coated as in example
3 and with pearlescent pigment coated as in example 4

| | Name | % (w/w) |
|---|---|---|
| Phase A | Synthetic Wax and Copernica Cernifera | 9.400 |
| | (carnauba) Wax | |
| | Stearoxymethicone/Dimethicone Copolymer | 0.500 |
| | Phenylpropyldimethylsiloxysilcate | 1.000 |
| | Polyhydroxystearic Acid | 0.700 |
| Phase B | Pentaerythrityl Tetra Di-t- | 0.100 |
| | Buthylhydroxyhdrocinnamate | |
| | Synthetic Wax | 4.560 |
| | Stearoxymethicone/Dimethicone Copolymer | 2.000 |
| | Polyethylene | 2.700 |
| Phase C | Disteardimonium Hectorite | 0.030 |
| | Propylene Carbonate | 0.010 |
| | Dicalcium Phosphate | 5.000 |
| | Pigment coated as in example 3 | 4.760 |
| Phase D | Silica | 2.600 |
| | Vinyl Dimethicone/MEthicone Silsesquioxane | 2.000 |
| | Crosspolymer | |
| | Pearlescent pigment coated as in example 4 | 18.450 |
| Phase E | Dimethicone | 46.190 |

### Example 9

Preparation of a shampoo with pearlescent
pigment coated as in example 4

| | Name | % (w/w) |
|---|---|---|
| Phase A | Water | 64.600 |
| | EDTA | 0.050 |
| | Water and Sodium Laureth Sulfate | 20.000 |
| Phase B | Water and Cocoamidopropyl Betaine | 8.000 |
| Phase C | Pearlescent pigment coated as in example 4 | 0.500 |
| Phase D | Glyceryl oleate | 0.300 |
| | Water and Coco-Glucoside | 2.000 |
| | Perfume | 0.300 |
| Phase E | Water and Acrylates Copolymer | 3.000 |
| Phase F | Water and Sodium Hydroxide | 0.200 |
| Phase G | Sodium Benzoate | 0.500 |
| Phase H | Citric Acid | 0.050 |
| Phase I | Sodium Chloride | 0.500 |

### Example 10

Preparation of a balm with pearlescent
pigment coated as in example 4

| | Name | % (w/w) |
|---|---|---|
| Phase A | Water | 86.150 |
| | EDTA | 0.050 |
| | Glyceryn | 1.000 |
| Phase B | Lactic Acid | 1.000 |
| Phase C | Pearlescent pigment coated as in example 4 | 0.800 |
| Phase D | Cetaryl Alcohol | 6.000 |
| | Stearamidopropyl Dimethylamine | 1.000 |
| | Behentrimonium Chloride and Dipropylen glycol | 1.300 |
| | Hydroxypropyl Cellulose | 0.400 |
| Phase E | Water and Pantenol | 0.500 |
| Phase F | Water and Sodium Hydroxide | 0.200 |
| Phase G | Perfume | 0.500 |
| | Phenoxyethanol | 0.700 |
| | Benzyl Alcohol | 0.400 |

1. Cosmetic composition containing at least one cosmetic powder with surface coating, in which said powder is characterized by comprising:
a) from 0.1% to 50% by weight of at least one chromophore selected among polymeric chromophores chemically linked to polyols with reactive functional groups;
b) from 99.9% to 50% by weight of a cosmetic powder or mixture of cosmetic powders having superficial reactive groups;
wherein said chromophore is chemically linked to the surface of said cosmetic powder or mixture of cosmetic powders.

2. Cosmetic composition according to claim 1, characterized in that said chromophore is selected among organic dies as azo groups, anthraquinones, indigoids, polymethines, cyanine, phthalocyanines, aryl-carbon, trialyl methane.

3. Cosmetic composition according to claim 1, characterized in that said reactive functional groups are selected among alkoxy groups, Si—H, carboxylic groups.

4. Cosmetic composition according to claim 1, characterized in that said cosmetic powder or mixture of cosmetic powders comprises a powder substrate having superficial hydroxylic groups functionalized to form stable covalent links with the chromophore.

5. Cosmetic composition according to claim 5, characterized in that said powder substrate is a pigment.

6. Cosmetic composition according to claim 6, characterized in that said pigment is a pearlescent pigment.

7. Cosmetic composition according to claim 1, characterized in that said polyols consist of polyethylene oxide.

8. Cosmetic composition according to claim 1, characterized in that said polyols consist of polypropylene oxide.

9. Cosmetic composition according to claim 1, characterized in that said polyols consist of polyesters.

10. Cosmetic composition according to claim 1, characterized in that said polyols consist of polybutylene oxide.

11. Cosmetic composition according to claim 1, characterized in that said polyols consist of polyglycerols.

12. Cosmetic composition according to claim 1, characterized in that said polyols consist of aliphatic polyols.

13. Cosmetic composition according to claim 1, characterized in that said polyols consist of polysiloxanes.

14. Cosmetic composition according to claim 1, characterized in that said polyols consist of polyols derived from sugar and polysaccharides.

15. Cosmetic composition according to claim 1, characterized in that said polyols consist of polycarbonates.

16. Cosmetic composition according to claim 1, characterized in that said polyols consist of polyacrylates.
